(19) United States
(12) Patent Application Publication
(10) Pub. No.: US 2010/0330592 A1
(43) Pub. Date: Dec. 30, 2010

(54) METHOD FOR DETECTING TRUNCATED MOLECULES

(76) Inventor: Marc E. Key, Ojai, CA (US)

Correspondence Address: KLARQUIST SPARKMAN, LLP, 121 SW SALMON STREET, SUITE 1600, PORTLAND, OR, 97204, US

(21) Appl. No.: 12/865,087

(22) PCT Filed: Jan. 29, 2009
(86) PCT No.: PCT/US2009/032431

§ 371 Date: Jul. 28, 2010

Related U.S. Application Data
(60) Provisional application No. 61/062,964, filed on Jan. 29, 2008.

(51) Int. Cl. G01N 33/53

(52) U.S. Cl. 435/7.21; 436/501; 435/7.9

(57) ABSTRACT

Exemplary disclosed embodiments may comprise, for example, providing a sample potentially comprising a native molecule and/or a truncated molecule. The native molecule comprises at least first and second regions recognized by first and second specific binding moieties, and the truncated molecule includes only one of the first and second regions. A composition comprising first and second specific binding moieties is applied to the sample in a manner effective to form first and second specific binding pairs with the first and second regions. For example, if the molecule is a protein, such as HER2, the protein may have a first epitope and a second epitope. Once a specific binding pair is formed, the pair must be visualized. Certain disclosed embodiments comprise a direct detection method whereby primary antibodies are coupled to signal generating moieties. Alternatively, signal amplification techniques can be used to visualize a specific binding pair.

### CROSS REFERENCE TO RELATED APPLICATION

This application claims the benefit of the earlier filing date of U.S. Provisional Application No. 61/062,964, filed on Jan. 29, 2008, which is incorporated herein by reference.

### FIELD

A method for detecting truncated molecules in a sample, particularly biological molecules, such as a proteins, and more particularly a method for dual detection of native and truncated proteins in a single sample, such as by using immunohistochemistry detection methods, is described.

### BACKGROUND

Protein analysis may require distinguishing between native protein and structurally altered proteins. A native protein is a protein as it occurs in its natural state, i.e. a protein having all amino acids encoded by an intact genetic sequence, unaltered by heat, chemicals, enzyme action or protocols used to extract the protein from a cell. Occasionally, a protein may be shorter than the native protein. For example, mutations may occur that cause the protein sequence to terminate prematurely during synthesis. These shortened proteins are called truncated proteins. The presence of truncated protein and no native protein, or certain ratios of truncated proteins to native proteins, may be diagnostic of particular pathologies. Detecting truncated proteins therefore is an increasingly important area in clinical diagnosis, including but not limited to the diagnosis of cancer and/or individuals disposed to cancer.

### A. Patent Literature

Truncated protein analysis is discussed in the patent literature. For example, U.S. Pat. No. 5,910,418 discusses truncated protein analysis, and states that:
In an alternative method for detecting APC mutations according to the present invention, immunohistochemistry or immunofluorescence is employed. A tissue sample or cell sample is prepared by any standard technique known in the art. Peripheral blood mononuclear cells can be fixed according to known techniques for immunochemical staining. Tissue samples can be frozen and embedded in a mounting compound, for example O.C.T. (Miles Laboratories). Thin sections can then be cut, incubated with an antibody specific for APC, stained, and observed. According to one method, an antibody which is immunoreactive with an epitope contained within the amino terminal portion of APC is used to stain a first sample of a tissue. A second antibody which is specifically immunoreactive with an epitope contained within the carboxy terminal portion of APC is used to stain a second sample of the same tissue. The amount of binding of each antibody is determined If substantially more of the amino terminal antibody binds to the tissue sample than does the carboxy terminal antibody, an APC truncation mutation is indicated. In one embodiment of the invention antibodies are used which are labelled with a detectable tag, such as an immunofluorescent compound or radioactive molecule. Such a detectable tag can also be attached to an additional antibody which is immunoreactive with the amino terminal or carboxy terminal anti-APC antibodies. In another embodiment of the invention an antibody is labelled by linkage to an enzyme which upon contact with an appropriate enzyme substrate, generates a colored reaction product. In still another embodiment of the invention detectable tags are linked to the antibody via an avidin:biotin ligand binding pair, as is generally known in the art. Typically, the antibody is linked to biotin and the detectable tag, e.g., alkaline phosphatase, is conjugated to avidin. According to one aspect of the invention the amino terminal and carboxy terminal antibodies are incubated with the same tissue sample or cell sample. In that case the antibodies are labelled with different detectable tags.
   And, according to U.S. Pat. No. 6,630,326:
RII mutations arising from addition or deletion of one or two A bases within the poly A sequences at nucleotides 709-718 encode truncated RII related proteins that contain the RII extracellular domain and that, lacking the RII membrane anchor, will be secreted from the cell into the blood. These truncated secreted mutant RII proteins can be concentrated and/or detected by immunoassays that either employ antibodies reactive with the external domain that is shared by both wild-type and mutant RII, and/or that employ antibodies reactive with mutant epitopes encoded at the carboxyl terminus of the truncated mutant forms of RII. For instance, secreted soluble mutant RII could be detected in blood by an ELISA that used antibodies reactive with the external domain that is shared by both wild-type and mutant RII, and/or that employed antibodies reactive with mutant epitopes encoded at the carboxyl terminus of the truncated mutant forms. In an alternate instance secreted soluble RII could be concentrated from blood by immuneprecipitation and then detected by western analysis. Again, this could be accomplished using antibodies reactive with the external domain that is shared by both wild-type and mutant RII, and/or by using antibodies reactive with mutant epitopes encoded at the carboxyl terminus of the truncated mutant forms.
Alternatively, the presence of mutant RII in a patient may be detected by immunoassays which detect production of an antibody response in the patient aimed against either the RII mutation or, due to breaking of immune tolerance, against other epitopes on the native RII protein. The provocation in a patient of a serologic antibody response to oncogenic mutation in a protein has been previously demonstrated in patients whose tumors harbor oncogenic mutations in the p53 protein (reviewed in Harris, et al., 1993, N. Eng. J. Med., 329:1318-1327). As frameshift mutations located within the ten base-pair poly A sequence at nucleotides 709-718 would be predicted to encode RII proteins truncated prior to their membrane spanning domain, it is expected such RII mutant proteins would be secreted directly into the blood, and would accordingly provoke a much more vigorous immune response than the mutations in the intracellular p53 protein discussed by Harris, et al. Such an immune response could provoke production of antibodies in the patient against epitopes in the mutant RII sequence or, as has been seen in some patients bearing tumors harboring p53 mutations, could result in breaking immune tolerance and so provoking an antibody response to epitopes on the native RII protein. Detection of a patient's serologic response to RII, using standard methods such as radioimmunoassay, or enzyme linked immunosorbant assay (ELISA), or other similar assays, is a routine matter for one skilled in the art.

As a final example, U.S. Patent Application No. 2003009231 discusses the protein truncation test (PTT), which detects nonsense and frameshift mutations that lead to truncated protein products. Genes associated with Duchenne muscular dystrophy, adenomatous polyposis coli, human mutL homologue and human nutS homologue (both involved in colon cancer), and BRAC1 (involved in familial breast cancer) can be screened for mutations in this manner, along with others. See Table 1 of U.S. Patent Application No. 2003009231. According to U.S. Patent Application No. 2003009231, the PTT technique typically involves producing a truncated protein using PCR and detecting the truncated protein using standard gel electrophoresis (e.g. SDS-PAGE) analysis combined with radioactive detection. U.S. Pat. Nos. 5,910,418, 6,630,326 and published application No. 2003009231 are incorporated herein by reference.

### B. Current Methods for Analyzing Truncated Proteins

Western blot analysis and single stain immunohistochemistry are the two general methods currently used for analyzing truncated proteins. Because truncated proteins are smaller than native proteins they can be analyzed by Western blot. Cells or tissues are homogenized and protein extracts are recovered. These proteins are then separated based on size using a gel. Size separated proteins are stained with an antibody that recognizes both the native and truncated form of the protein. Although the antibody alone cannot distinguish between native and truncated forms, the antibody information along with the size information can be used to distinguish native and truncated forms. Western blot analysis is laborious, expensive, and highly complex, and as a result is not routinely used in most clinical laboratories.

Single stain immunohistochemistry involves staining proteins without extraction; instead, the proteins are stained in situ (in the tissue). This method requires at least two antibodies and two different tissue sample preparations. A small piece of the same tissue is placed on two different microscope slides. Slide 1 is stained with the first antibody and slide 2 is stained with the second antibody. A pathologist reviews the stains under a microscope, identifies which cells and structures of slide 1 are stained, and compares this staining pattern to the staining pattern of slide 2, often simply by memorizing the staining patterns and mentally comparing the two. A modification of this method is to use a special microscope that displays images from two different slides side-by-side on a monitor. This makes comparisons between two slides much easier. Another modification involves taking digital images of two slides and then comparing the two images side-by-side on a computer display. The disadvantage of this method is that it requires more tissue, is twice as expensive, and requires twice as much labor. Secondly the comparison between the two slides requires special training.

As a result, despite these known technologies, there still is a need for a method for distinguishing truncated proteins from native proteins.

### SUMMARY

The present invention concerns a method for detecting a truncated form of a molecule in a sample. Certain embodiments of the method involve double staining techniques for identifying both a native form and the truncated form, if present, in the same sample.

Exemplary embodiments use the method to analyze biological molecules, such as truncated proteins. Examples of truncated proteins include, but are not limited to: growth factors, such as Epidermal Growth Factor Receptor (EGFR, HER1, HER2, HER2/neu), as truncated forms of EGFR have been implicated in several types of cancers, including breast cancer and glioblastoma brain tumors; Parkinson disease, which may result from a mutation in the parkin protein, as in a mouse model of Parkinson disease the parkin mutation results in a truncated parkin protein; Crohn's Disease, as three main NOD2 mutations result in a truncated protein that predisposes affected individuals to Crohn's disease; BRCA1 mutations, which account for 50% of all familial, early-onset female breast cancers, and some of these mutations result in truncated BRCA1 protein; and cardiac sarcoidosis, which has been associated with the expression of a splice variant coding for a truncated BTNL2 protein. Several other truncated oncoproteins have been identified, but the significance of these truncated forms is currently unknown. Some of the genes transcribing these oncoproteins are cph, fos, bax, bid, and ras.

The method may comprise, for example, providing a sample potentially comprising a native molecule and/or a truncated molecule. The native molecule comprises at least first and second regions recognized by first and second specific binding moieties, and the truncated molecule includes only one of the first and second regions. A composition comprising the first and second specific binding moieties is applied to the sample in a manner effective to form first and second specific binding pairs with the first and second regions.

For example, if the molecule is a protein, the protein may have a first epitope and a second epitope. Certain disclosed embodiments comprise treating the sample with at least two primary antibodies in a manner effective to form epitope-antibody complexes. A first primary antibody recognizes a first epitope on a truncated molecule and a second primary antibody recognizes a second epitope on the native molecule and on the truncated molecule. The antibodies may be polyclonal, but more typically are monoclonal antibodies from different species, including but not limited to mouse and/or rabbit monoclonal antibodies.

Once a specific binding pair is formed, the pair must be visualized. Certain disclosed embodiments comprise a direct detection method whereby primary antibodies are coupled to signal generating moieties. Alternatively, signal amplification techniques can be used to visualize a specific binding pair. Certain disclosed embodiments comprise treating the sample with first and second secondary antibodies that recognize the first and second primary antibodies. For example, the method may comprise treating the sample with first and second primary monoclonal antibodies from a first species. In these situations, the method may comprise treating the first and second primary antibodies that are bound to epitopes with first and second secondary monoclonal anti-antibodies that specifically bind to the first and second primary antibodies. The first and second secondary anti-antibodies are coupled to signal generating moieties.

As yet another visualization method, antibodies, such as the first and second primary antibodies, may have at least one hapten, and typically plural haptens, conjugated thereto. The method then further comprises treating the sample with anti-hapten antibodies. These anti-hapten antibodies may be effectively coupled to signal generating moieties, such as enzymes, chromophores, quantum dots, or combinations thereof. For certain exemplary embodiments, the signal generating moieties are enzymes, such as peroxidase, alkaline phosphatase, acid phosphatase, glucose oxidase, β-galactosidase, β-glucuronidase or β-lactamase. Where the signal generating moieties are enzymes, the method further comprises treating the sample with a first substrate for the first enzyme and a second substrate for the second enzyme to produce detectable reactions or detectable products.

Various modifications to this general method also are contemplated. For example, the method may further comprise pre-treating the sample, such as by using antigen retrieval, to facilitate reaction with primary antibodies. The method also may further comprise blocking or substantially eliminating endogenous enzyme or enzymes that potentially interfere with the analysis. The method also may further comprise counterstaining the sample. Certain disclosed embodiments can be automated, in whole or in part. And, the method may further comprise using computer analysis and/or an image analysis system.

The present method is particularly exemplified by reference to a method for detecting a truncated HER2 protein. The HER2 protein includes an external domain having a first epitopic region solely present on a native HER2 protein, and an internal domain having an epitopic portion that is present on both the native protein and on a truncated HER2 protein. A tissue sample is provided that potentially includes a native HER2 protein, a truncated HER2 protein, or both. The tissue sample is incubated with at least two primary antibodies. A first antibody recognizes the first epitope on the native HER2 protein. A second antibody recognizes the second epitope on both the native HER2 protein and the truncated form of the HER2 protein. The sample is treated with first and second secondary antibodies that specifically bind to the first and second primary antibodies, the first and second secondary antibodies being effectively coupled to enzymes useful as signal generating moieties. After formation of the specific binding pairs, suitable enzyme substrates are applied, and color changes are monitored to determine if native HER2 is present, truncated HER2 is present, or both forms are present. For particular embodiments, the enzymes are peroxidase and alkaline phosphatase, and the substrates are diaminobenzidine as a substrate for peroxidase, which produces a chocolate color reaction, and Fast Red as a substrate for alkaline phosphatase, which produces a red color. Again with reference to particular embodiments, the first primary antibody is rabbit monoclonal antibody, clone SP3, to the HER2 external domain The second primary antibody is mouse monoclonal antibody, clone SPM172, to the HER2 internal domain.

Secondary antibodies may be provided as polymer conjugates. For example, a first polymer conjugate may comprise a polymer backbone, anti-rabbit Ig secondary antibodies, and peroxidase. A second polymer conjugate may comprise a polymer backbone, anti-mouse Ig secondary antibodies, and alkaline phosphatase.

Test kits also are disclosed. One embodiment of a disclosed test kit useful for detecting truncated proteins comprises at least two specific binding moieties. A first specific binding moiety is provided for detecting a first specific binding pair on a native form of a protein. A second specific binding moiety is provided for detecting a second binding pair on both the native form of the protein and on a truncated form of the protein. The first and second specific binding moieties may be first and second primary antibodies. Disclosed test kits also may include first and second secondary anti-primary antibodies. The first and second secondary anti-primary antibodies may be coupled to signal generating moieties. If the signal generating moieties are enzymes, then the test kit may further include substrates for reaction with the enzymes. Alternatively, the first and second primary antibodies may have at least one hapten conjugated thereto. If so, the test kit may further comprise anti-hapten antibodies.

A particular embodiment of a disclosed test kit is useful for detecting truncated HER2 proteins. Such test kits typically comprise at least two primary antibodies. A first primary antibody recognizes a first epitope on a native HER2 protein. A second primary antibody recognizes a second epitope on both the native HER2 protein and the truncated form of the HER2 protein. An antigen retrieval composition optionally may be included in the test kit. First and second secondary antibodies are provided that specifically bind to the first and second primary antibodies. The first and second secondary antibodies are effectively coupled to enzymes useful as signal generating moieties. As a result, the test kit typically includes suitable enzyme substrates too, and also optionally may include a counterstain.

The foregoing and other objects, features, and advantages of the invention will become more apparent from the following detailed description, which proceeds with reference to the accompanying figures.

### BRIEF DESCRIPTION OF THE DRAWINGS

FIG. 1 is a schematic drawing illustrating one embodiment of a method for detecting a native protein form and a truncated protein form using first and second primary antibodies that recognize first and second epitopes.

FIG. 2 is a schematic drawing illustrating one embodiment of a method for detecting a native protein form and a truncated protein form using signal generating moieties coupled to first and second primary antibodies that recognize first and second epitopes.

FIG. 3 is a schematic drawing illustrating one embodiment of a method for using enzymes and enzyme substrates as signal generating moieties to generate a detectable product.

FIG. 4 is a schematic drawing illustrating a signal amplification process using anti-antibodies coupled to signal generating moieties for detecting two or more epitopes bound to primary antibodies.

FIG. 5 is a schematic diagram illustrating one embodiment of a method for amplifying detection signals using haptens coupled to primary antibodies and anti-hapten antibodies coupled to enzymes useful as signal generating moieties.

### DETAILED DESCRIPTION

### I. Terms and Introduction

Unless otherwise noted, technical terms are used according to conventional usage. Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar references.

As used herein, the singular terms “a,” “an,” and “the” include plural referents unless context clearly indicates otherwise. Similarly, the word “or” is intended to include “and” unless the context clearly indicates otherwise. Also, as used herein, the term “comprises” means “includes.” Hence “comprising A or B” means including A, B, or A and B. It is further to be understood that all nucleotide sizes or amino acid sizes, and all molecular weight or molecular mass values, given for nucleic acids or polypeptides or other compounds are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

In order to facilitate review of the various examples of this disclosure, the following explanations of specific terms are provided:

Amplification: Certain embodiments of the present invention allow a single target to be detected using plural visualization complexes, where the complexes can be the same or different, to facilitate identification and/or quantification of a particular target.

Analog, Derivative or Mimetic: An analog is a molecule that differs in chemical structure from a parent compound, for example a homolog (differing by an increment in the chemical structure, such as a difference in the length of an alkyl chain), a molecular fragment, a structure that differs by one or more functional groups, a change in ionization. Structural analogs are often found using quantitative structure activity relationships (QSAR), with techniques such as those disclosed in Remington (The Science and Practice of Pharmacology, 19th Edition (1995), chapter 28). A derivative is a biologically active molecule derived from the base structure. A mimetic is a molecule that mimics the activity of another molecule, such as a biologically active molecule. Biologically active molecules can include chemical structures that mimic the biological activities of a compound.

Animal: Refers to living, multi-cellular vertebrate organisms, a category that includes, for example, mammals and birds.

Antibody: “Antibody” collectively refers to immunoglobulins or immunoglobulin-like molecules (including by way of example and without limitation, IgA, IgD, IgE, IgG and IgM, combinations thereof, and similar molecules produced during an immune response in any vertebrate, for example, in mammals such as humans, goats, rabbits and mice) and antibody fragments that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample.

More particularly, “antibody” refers to a polypeptide ligand comprising at least a light chain or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody.

This includes intact immunoglobulins and the variants and portions of them well known in the art. Antibody fragments include proteolytic antibody fragments [such as F(ab′)₂ fragments, Fab′ fragments, Fab′-SH fragments and Fab fragments as are known in the art], recombinant antibody fragments (such as sFv fragments, dsFv fragments, bispecific sFv fragments, bispecific dsFv fragments, F(ab)′₂ fragments, single chain Fv proteins (“scFv”), disulfide stabilized Fv proteins (“dsFv”), diabodies, and triabodies (as are known in the art), and camelid antibodies (see, for example, U.S. Pat. Nos. 6,015,695; 6,005,079-5,874,541; 5,840,526; 5,800,988; and 5,759,808). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

Typically, a naturally occurring immunoglobulin has heavy (H) chains and light (L) chains interconnected by disulfide bonds. There are two types of light chain, lambda (λ) and kappa (k). There are five main heavy chain classes (or isotypes) which determine the functional activity of an antibody molecule: IgM, IgD, IgG, IgA and IgE.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds RET will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

Antigen: A compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, nucleic acids and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens. In one example, an antigen is a Bacillus antigen, such as γPGA.

Avidin: Any type of protein that specifically binds biotin to the substantial exclusion of other small molecules that might be present in a biological sample. Examples of avidin include avidins that are naturally present in egg white, oilseed protein (e.g., soybean meal), and grain (e.g., corn/maize) and streptavidin, which is a protein of bacterial origin.

Binding affinity: The tendency of one molecule to bind (typically non-covalently) with another molecule, such as the tendency of a member of a specific binding pair for another member of a specific binding pair. A binding affinity can be measured as a binding constant, which binding affinity for a specific binding pair (such as an antibody/antigen pair or nucleic acid probe/nucleic acid sequence pair) can be at least 1×10⁵ M⁻¹, such as at least 1×10⁶ M⁻¹, at least 1×10⁷ M⁻¹ or at least 1×108 M⁻¹. In one embodiment, binding affinity is calculated by a modification of the Scatchard method described by Frankel et al., Mol. Immunol., 16:101-106, 1979. In another embodiment, binding affinity is measured by an antigen/antibody dissociation rate. In yet another embodiment, a high binding affinity is measured by a competition radioimmunoassay. In several examples, a high binding affinity for an antibody/antigen pair is at least about 1×10⁸ M⁻¹. In other embodiments, a high binding affinity is at least about 1.5×10⁸ M⁻¹, at least about 2.0×10⁸ M⁻¹, at least about 2.5×10⁸ M⁻¹, at least about 3.0×10⁸ M⁻¹, at least about 3.5×10⁸ M⁻¹, at least about 4.0×10⁸ M⁻¹, at least about 4.5×10⁸ M⁻¹, or at least about 5.0×10⁸ M⁻¹.

Carrier: A molecule to which a hapten or an antigen can be bound. Carrier molecules include immunogenic carriers and specific-binding carriers. When bound to an immunogenic carrier, the bound molecule may become immunogenic. Immunogenic carriers may be chosen to increase the immunogenicity of the bound molecule and/or to elicit antibodies against the carrier, which are diagnostically, analytically, and/or therapeutically beneficial. Covalent linking of a molecule to a carrier can confer enhanced immunogenicity and T-cell dependence (Pozsgay et al., PNAS 96:5194-97, 1999; Lee et al., J. Immunol. 116:1711-18, 1976; Dintzis et al., PNAS 73:3671-75, 1976). Useful carriers include polymeric carriers, which can be natural (for example, proteins from bacteria or viruses), semi-synthetic or synthetic materials containing one or more functional groups to which a reactant moiety can be attached. Specific binding carriers can by any type of specific binding moiety, including an antibody, a nucleic acid, an avidin, a protein-nucleic acid.

Chimeric antibody: An antibody that has framework residues from one species, such as human, and CDRs (which generally confer antigen binding) from another species, such as a murine antibody that specifically binds RET.

Conjugating, joining, bonding or linking: Covalently linking one molecule to another molecule to make a larger molecule. Refers to, for example, making two polypeptides into one contiguous polypeptide molecule, or to covalently attaching a hapten or other molecule to a polypeptide, such as an scFv antibody. In the specific context, the terms include joining a ligand, such as an antibody moiety, to an effector molecule (“EM”). The linkage can be either by chemical or recombinant means. “Chemical means” refers to a reaction between the antibody moiety and the effector molecule such that there is a covalent bond formed between the two molecules to form one molecule.

Coupled: When referring to a first atom or molecule being “coupled” to a second atom or molecule, “coupled” can be both directly coupled and indirectly coupled. A secondary antibody provides an example of indirect coupling. One specific example of indirect coupling is a rabbit anti-hapten primary antibody that is bound by a mouse anti-rabbit IgG antibody, that is in turn bound by a goat anti-mouse IgG antibody that is covalently linked to a signal generating moiety.

Epitope: An antigenic determinant These are particular chemical groups or contiguous or non-contiguous peptide sequences on a molecule that are antigenic, that is, that elicit a specific immune response. An antibody binds a particular antigenic epitope.

Hapten: A molecule, typically a relatively small molecule, that can combine specifically with an antibody, but typically is substantially incapable of being immunogenic except in combination with a carrier molecule.

Homopolymer: This term refers to a polymer formed by the bonding together of multiple units of a single type of molecular species, such as a single monomer (for example, an amino acid).

Humanized antibody: An antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by genetic engineering (see, for example, U.S. Pat. No. 5,585,089).

Humanized immunoglobulin: Refers to an immunoglobulin that includes a human framework region and one or more CDRs from a non-human (for example a mouse, rat, or synthetic) immunoglobulin. The non-human immunoglobulin providing the CDRs is termed a “donor,” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences.

Inhibiting or Treating a Disease: Inhibiting the full development of a disease or condition, for example, in a subject who is at risk for a disease. “Treatment” refers to a therapeutic intervention that ameliorates a sign or symptom of a disease or pathological condition after it has begun to develop. As used herein, the term “ameliorating,” with reference to a disease, pathological condition or symptom, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease.

Isolated: An “isolated” microorganism (such as a virus, bacterium, fungus, or protozoan) has been substantially separated or purified away from microorganisms of different types, strains, or species. Microorganisms can be isolated by a variety of techniques, including serial dilution and culturing.

An “isolated” biological component (such as a nucleic acid molecule, protein or organelle) has been substantially separated or purified away from other biological components in the cell of the organism in which the component naturally occurs, such as other chromosomal and extra-chromosomal DNA and RNA, proteins, and organelles. Nucleic acids and proteins that have been “isolated” include nucleic acids and proteins purified by standard purification methods. The term also embraces nucleic acids and proteins prepared by recombinant expression in a host cell, as well as chemically synthesized nucleic acids or proteins, or fragments thereof.

Linker peptide: A peptide within an antibody binding fragment (such as an Fv fragment) which serves to indirectly bond the variable heavy chain to the variable light chain. “Linker” can also refer to a peptide serving to link a targeting moiety, such as a scFv, to an effector molecule, such as a cytotoxin or a signal generating moiety.

Mammal: This term includes both human and non-human mammals.

Molecule of interest or Target: A molecule for which the presence, location and/or concentration is to be determined Examples of molecules of interest include proteins and nucleic acid sequences tagged with haptens.

Monoclonal antibody: An antibody produced by a single clone of B-lymphocytes or by a cell into which the light and heavy chain genes of a single antibody have been transfected. Monoclonal antibodies are produced by methods known to those of ordinary skill in the art, for instance by making hybrid antibody-forming cells from a fusion of myeloma cells with immune spleen cells. Monoclonal antibodies include humanized monoclonal antibodies.

Multiplex, -ed, -ing: Embodiments of the present invention allow multiple targets in a sample to be detected substantially simultaneously, or sequentially, as desired, using plural different conjugates. Multiplexing can include identifying and/or quantifying nucleic acids generally, DNA, RNA, peptides, proteins, both individually and in any and all combinations. Multiplexing also can include detecting two or more of a gene, a messenger and a protein in a cell in its anatomic context.

Nanoparticle: A nanoscale particle with a size that is measured in nanometers, for example, a nanoscopic particle that has at least one dimension of less than about 100 nm. Examples of nanoparticles include paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes and quantum dots. A nanoparticle can produce a detectable signal, for example, through absorption and/or emission of photons (including radio frequency and visible photons) and plasmon resonance.

Neoplasia and Tumor: The process of abnormal and uncontrolled growth of cells. Neoplasia is one example of a proliferative disorder.

The product of neoplasia is a neoplasm (a tumor), which is an abnormal growth of tissue that results from excessive cell division. A tumor that does not metastasize is referred to as “benign.” A tumor that invades the surrounding tissue and/or can metastasize is referred to as “malignant.” Examples of hematological tumors include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancer, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma).

Polypeptide: A polymer in which the monomers are amino acid residues joined together by amide bonds. When the amino acids are alpha-amino acids, either the L-optical isomer or the D-optical isomer can be used. The terms “polypeptide” or “protein” as used herein are intended to encompass any amino acid sequence, and include modified sequences, such as glycoproteins. The term “polypeptide” is specifically intended to cover naturally occurring proteins, as well as those produced recombinantly or synthetically.

The term “residue” or “amino acid residue” includes reference to an amino acid that is incorporated into a protein, polypeptide, or peptide.

Protein: A molecule, particularly a polypeptide, comprised of amino acids.

Purified: The term “purified” does not require absolute purity; rather, it is intended as a relative term. Thus, for example, a purified peptide, protein, conjugate, or other active compound is one that is isolated in whole or in part from proteins or other contaminants. Generally, substantially purified peptides, proteins, conjugates, or other active compounds for use within the disclosure comprise more than 80% of all macromolecular species present in a preparation prior to admixture or formulation of the peptide, protein, conjugate or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, conjugate or other active compound is purified to represent greater than 90%, often greater than 95%, of all macromolecular species present in a purified preparation prior to admixture with other formulation ingredients. In other cases, the purified preparation may be essentially homogeneous, wherein other macromolecular species are not detectable by conventional techniques.

Quantum dot: A nanoscale particle that exhibits size-dependent electronic and optical properties due to quantum confinement. Quantum dots have, for example, been constructed of semiconductor materials (e.g., cadmium selenide and lead sulfide) and from crystallites (grown via molecular beam epitaxy), etc. A variety of quantum dots having various surface chemistries and fluorescence characteristics are commercially available from Invitrogen Corporation, Eugene, Oreg. (see, for example, U.S. Pat. Nos. 6,815,064, 6,682,596 and 6,649,138, each of which is incorporated by reference). Quantum dots are also commercially available from Evident Technologies (Troy, N.Y.). Other quantum dots include alloy quantum dots such as ZnSSe, ZnSeTe, ZnSTe, CdSSe, CdSeTe, ScSTe, HgSSe, HgSeTe, HgSTe, ZnCdS, ZnCdSe, ZnCdTe, ZnHgS, ZnHgSe, ZnHgTe, CdHgS, CdHgSe, CdHgTe, ZnCdSSe, ZnHgSSe, ZnCdSeTe, ZnHgSeTe, CdHgSSe, CdHgSeTe, InGaAs, GaAlAs, and InGaN quantum dots. Alloy quantum dots and methods for making the same are disclosed, for example, in U.S. Patent Application Publication No. 2005/0012182 and PCT Publication WO 2005/001889, which are incorporated herein by reference.

Sample: A biological specimen containing genomic DNA, RNA (including mRNA), protein, or combinations thereof, obtained from a subject. Examples include, but are not limited to, peripheral blood, urine, saliva, tissue biopsy, surgical specimen, amniocentesis samples and autopsy material. In one example, a sample includes a biopsy of an adenocarcinoma, a sample of noncancerous tissue, a sample of normal tissue (from a subject not afflicted with a known disease or disorder).

Specific binding moiety: A member of a specific-binding pair. Specific binding pairs are pairs of molecules that bind each other to the substantial exclusion of binding to other molecules (for example, specific binding pairs can have a binding constant that is at least 10³ M⁻¹ greater, 10⁴ M⁻¹ greater or 10⁵ M⁻¹ greater than a binding constant for either of the two members of the binding pair with other molecules in a biological sample). Particular examples of specific binding moieties include specific binding proteins (for example, antibodies, lectins, avidins such as streptavidins, and protein A), nucleic acids sequences, and protein-nucleic acids. Specific binding moieties can also include the molecules (or portions thereof) that are specifically bound by such specific binding proteins.

Subject: The term “subject” includes both human and veterinary subjects, for example, humans, non-human primates, dogs, cats, horses, and cows.

### II. General Description of Method

### A. Target Molecule

Disclosed embodiments are generally applicable for analyzing any molecule to determine whether it exists in a native form or a truncated form. For certain embodiments, the target molecule is a biological molecule, and even more specifically is a biological molecule that expresses at least two epitopes recognized by a specific binding pair, such as primary antibodies. A first epitope is present on the truncated protein, whereas the native molecule has both the first and a second epitope.

### B. Target Proteins

While the disclosed embodiments are generally useful for distinguishing between a first molecular form, often referred to as a native form, and a second truncated form, the invention is particularly useful for target peptides, polypeptides or proteins having at least two distinguishable epitopes (such targets may be collectively referred to herein as “protein”). In particular examples the target protein is produced from a genomic target sequence or genomic subsequence, for example from a eukaryotic genome, such as a human genome. In some examples, the target protein is produced from a nucleic acid molecule selected from a pathogen, such as a virus, bacteria, or intracellular parasite, such as from a viral genome. For example, the target protein may be produced from a nucleic acid sequence associated with (e.g., correlated with, causally implicated in, etc.) a disease. In certain examples, the selected target protein is produced from a nucleic acid molecule associated with a neoplastic disease (or cancer). For example, the nucleic acid sequence can include at least one gene associated with cancer [e.g., HER2 (human epidermal growth factor receptor-2), c-Myc, n-Myc, Abl, Bcl2, Bcl6, Rb1, p53, EGFR, TOP2A, MET, or genes encoding other receptors and/or signaling molecules, etc.] or chromosomal region associated with a cancer. The target protein can be produced from a nucleic acid sequence associated with a truncation that has been correlated with a cancer.

The target protein can be produced from a nucleic acid sequence that can vary substantially in size. Without limitation, the nucleic acid sequence can have a variable number of base pairs, such as at least 1000 base pairs in length, at least 50,000, at least 100,000, or even at least 250,000, 500,000, or several million (e.g., at least 3 million) base pairs in overall length. In some examples, a target molecule is selected that is associated with a disease or condition, such that detection of protein truncation can be used to infer information (such as diagnostic or prognostic information for the subject from whom the sample is obtained) relating to the disease or condition. In a specific example, the target peptide, polypeptide or protein sequence is produced from a genomic target nucleic acid sequence, such as a mammalian or viral genomic sequence.

In examples where the target protein is produced by a eukaryotic genome (such as a mammalian genome, e.g., a human genome), the target protein typically represents a small portion of the total genomic product (or a small portion of a single chromosome) of the organism (for example, protein produced from less than 20%, less than 10%, less than 5%, less than 2%, or less than 1% of the genomic DNA (or a single chromosome) of the organism). In some examples where the target protein is produced by a sequence (e.g., genomic target nucleic acid sequence) from an infectious organism (such as a virus), the target protein sequence can represent a larger proportion (for example, 50% or more) or even all of the genome of the infectious organism.

In specific, non-limiting examples, a target protein is produced by a nucleic acid sequence (e.g., genomic target nucleic acid sequence) associated with a neoplasm (for example, a cancer). Numerous chromosome abnormalities (including translocations and other rearrangements, reduplication or deletion) have been identified in neoplastic cells, especially in cancer cells, such as B cell and T cell leukemias, lymphomas, breast cancer, colon cancer, neurological cancers and the like. Therefore, in some examples, at least a portion of the target molecule is produced by a nucleic acid sequence (e.g., genomic target nucleic acid sequence) reduplicated or deleted in at least a subset of cells in a sample.

Oncogenes are known to be responsible for several human malignancies. For example, chromosomal rearrangements involving the SYT gene located in the breakpoint region of chromosome 18q11.2 are common among synovial sarcoma soft tissue tumors. The t(18q11.2) translocation can be identified, for example, using probes with different labels: the first probe includes FPC nucleic acid molecules generated from a target nucleic acid sequence that extends distally from the SYT gene, and the second probe includes FPC nucleic acid generated from a target nucleic acid sequence that extends 3′ or proximal to the SYT gene. When probes corresponding to these target nucleic acid sequences (e.g., genomic target nucleic acid sequences) are used in an in situ hybridization procedure, normal cells, which lack a t(18q11.2) in the SYT gene region, exhibit two fusion (generated by the two labels in close proximity) signals, reflecting the two intact copies of SYT. Abnormal cells with a t(18q11.2) exhibit a single fusion signal.

Numerous examples of reduplication of genes involved in neoplastic transformation have been observed. Truncated proteins produced from such a nucleic acid sequence (e.g., genomic target nucleic acid sequence) included in a gene (e.g., an oncogene) that is reduplicated in one or more malignancies (e.g., a human malignancy) can be detected using the present method. For example, HER2, also known as c-erbB2 or HER2/neu, is a gene that plays a role in the regulation of cell growth (a representative human HER2 genomic sequence is provided at GENBANK™ Accession No. NC_{—}000017, nucleotides 35097919-35138441). The gene codes for a 185 kd, transmembrane, cell surface receptor that is a member of the tyrosine kinase family. HER2 is amplified in human breast, ovarian, and other cancers. Therefore, truncated proteins produced from a HER2 gene (or a region of chromosome 17 that includes the HER2 gene) can be detected using the presently disclosed method.

In other examples, a target protein produced from a nucleic acid sequence (e.g., genomic target nucleic acid sequence) is selected that is a tumor suppressor gene that is deleted (lost) in malignant cells. For example, the p16 region (including D9S1749, D9S1747, p16(INK4A), p14(ARF), D9S1748, p15(INK4B), and D9S 1752) located on chromosome 9p21 is deleted in certain bladder cancers. Chromosomal deletions involving the distal region of the short arm of chromosome 1 (that encompasses, for example, SHGC57243, TP73, EGFL3, ABL2, ANGPTL1, and SHGC-1322), and the pericentromeric region (e.g., 19p13-19q13) of chromosome 19 (that encompasses, for example, MAN2B1, ZNF443, ZNF44, CRX, GLTSCR2, and GLTSCR1)) are characteristic molecular features of certain types of solid tumors of the central nervous system.

The aforementioned examples are provided solely for purpose of illustration and are not intended to be limiting. Numerous other cytogenetic abnormalities that correlate with neoplastic transformation and/or growth are known to those of skill in the art. Target proteins that are produced by nucleic acid sequences (e.g., genomic target nucleic acid sequences), which have been correlated with neoplastic transformation and which are useful in the disclosed methods, also include the EGFR gene (7p12; e.g., GENBANK™ Accession No. NC_{—}000007, nucleotides 55054219-55242525), the C-MYC gene (8q24.21; e.g., GENBANK™ Accession No. NC_{—}000008, nucleotides 128817498-128822856), D5S271 (5p15.2), lipoprotein lipase (LPL) gene (8p22; e.g., GENBANK™ Accession No. NC_{—}000008, nucleotides 19841058-19869049), RB1 (13q14; e.g., GENBANK™ Accession No. NC_{—}000013, nucleotides 47775912-47954023), p53 (17p13.1; e.g., GENBANK™ Accession No. NC_{—}000017, complement, nucleotides 7512464-7531642)), N-MYC (2p24; e.g., GENBANK™ Accession No. NC_{—}000002, complement, nucleotides 151835231-151854620), CHOP (12q13; e.g., GENBANK™ Accession No. NC_{—}000012, complement, nucleotides 56196638-56200567), FUS (16p11.2; e.g., GENBANK™ Accession No. NC_{—}000016, nucleotides 31098954-31110601), FKHR (13p14; e.g., GENBANK™ Accession No. NC_{—}000013, complement, nucleotides 40027817-40138734), as well as, for example: ALK (2p23; e.g., GENBANK™ Accession No. NC_{—}000002, complement, nucleotides 29269144-29997936), Ig heavy chain, CCND1 (11q13; e.g., GENBANK™ Accession No. NC_{—}000011, nucleotides 69165054..69178423), BCL2 (18q21.3; e.g., GENBANK™ Accession No. NC_{—}000018, complement, nucleotides 58941559-59137593), BCL6 (3q27; e.g., GENBANK™ Accession No. NC_{—}000003, complement, nucleotides 188921859-188946169), MALF1, AP1 (1p32-p31; e.g., GENBANK™ Accession No. NC_{—}000001, complement, nucleotides 59019051-59022373), TOP2A (17g21-q22; e.g., GENBANK™ Accession No. NC_{—}000017, complement, nucleotides 35798321-35827695), TMPRSS (21q22.3; e.g., GENBANK™ Accession No. NC_{—}000021, complement, nucleotides 41758351-41801948), ERG (21q22.3; e.g., GENBANK™ Accession No. NC_{—}000021, complement, nucleotides 38675671-38955488); ETV1 (7p21.3; e.g., GENBANK™ Accession No. NC_{—}000007, complement, nucleotides 13897379-13995289), EWS (22q12.2; e.g., GENBANK™ Accession No. NC_{—}000022, nucleotides 27994271-28026505); FLI1 (11q24.1-q24.3; e.g., GENBANK™ Accession No. NC_{—}000011, nucleotides 128069199-128187521), PAX3 (2q35-q37; e.g., GENBANK™ Accession No. NC_{—}000002, complement, nucleotides 222772851-222871944), PAX7 (1p36.2-p36.12; e.g., GENBANK™ Accession No. NC_{—}000001, nucleotides 18830087-18935219, PTEN (10q23.3; e.g., GENBANK™ Accession No. NC_{—}000010, nucleotides 89613175-89716382), AKT2 (19q13.1-q13.2; e.g., GENBANK™ Accession No. NC_{—}000019, complement, nucleotides 45431556-45483036), MYCL1 (1p34.2; e.g., GENBANK™ Accession No. NC_{—}000001, complement, nucleotides 40133685-40140274), REL (2p13-p12; e.g., GENBANK™ Accession No. NC_{—}000002, nucleotides 60962256-61003682) and CSF1R (5q33-q35; e.g., GENBANK™ Accession No. NC_{—}000005, complement, nucleotides 149413051-149473128).

In other examples, a target protein is selected from a virus or other microorganism associated with a disease or condition. Detection of the virus- or microorganism-derived target nucleic acid sequence (e.g., genomic target nucleic acid sequence) in a cell or tissue sample is indicative of the presence of the organism. For example, the target peptide, polypeptide or protein can be selected from the genome of an oncogenic or pathogenic virus, a bacterium or an intracellular parasite (such as Plasmodium falciparum and other Plasmodium species, Leishmania (sp.), Cryptosporidium parvum, Entamoeba histolytica, and Giardia lamblia, as well as Toxoplasma, Eimeria, Theileria, and Babesia species).

In some examples, the target protein is produced from a nucleic acid sequence (e.g., genomic target nucleic acid sequence) from a viral genome. Exemplary viruses and corresponding genomic sequences (GENBANK™ RefSeq Accession No. in parentheses) include human adenovirus A (NC_{—}001460), human adenovirus B (NC_{—}004001), human adenovirus C (NC_{—}001405), human adenovirus D (NC_{—}002067), human adenovirus E (NC_{—}003266), human adenovirus F (NC_{—}001454), human astrovirus (NC_{—}001943), human BK polyomavirus (V01109; GI:60851) human bocavirus (NC_{—}007455), human coronavirus 229E (NC_{—}002645), human coronavirus HKU1 (NC_{—}006577), human coronavirus NL63 (NC_{—}005831), human coronavirus OC43 (NC_{—}005147), human enterovirus A (NC_{—}001612), human enterovirus B (NC_{—}001472), human enterovirus C (NC_{—}001428), human enterovirus D (NC_{—}001430), human erythrovirus V9 (NC_{—}004295), human foamy virus (NC_{—}001736), human herpesvirus 1 (Herpes simplex virus type 1) (NC_{—}001806), human herpesvirus 2 (Herpes simplex virus type 2) (NC_{—}001798), human herpesvirus 3 (Varicella zoster virus) (NC_{—}001348), human herpesvirus 4 type 1 (Epstein-Barr virus type 1) (NC_{—}007605), human herpesvirus 4 type 2 (Epstein-Barr virus type 2) (NC_{—}009334), human herpesvirus 5 strain AD169 (NC_{—}001347), human herpesvirus 5 strain Merlin Strain (NC_{—}006273), human herpesvirus 6A (NC_{—}001664), human herpesvirus 6B (NC_{—}000898), human herpesvirus 7 (NC_{—}001716), human herpesvirus 8 type M (NC_{—}003409), human herpesvirus 8 type P (NC_{—}009333), human immunodeficiency virus 1 (NC_{—}001802), human immunodeficiency virus 2 (NC_{—}001722), human metapneumovirus (NC_{—}004148), human papillomavirus-1 (NC_{—}001356), human papillomavirus-18 (NC_{—}001357), human papillomavirus-2 (NC_{—}001352), human papillomavirus-54 (NC_{—}001676), human papillomavirus-61 (NC_{—}001694), human papillomavirus-cand90 (NC_{—}004104), human papillomavirus RTRX7 (NC_{—}004761), human papillomavirus type 10 (NC_{—}001576), human papillomavirus type 101 (NC_{—}008189), human papillomavirus type 103 (NC_{—}008188), human papillomavirus type 107 (NC_{—}009239), human papillomavirus type 16 (NC_{—}001526), human papillomavirus type 24 (NC_{—}001683), human papillomavirus type 26 (NC_{—}001583), human papillomavirus type 32 (NC_{—}001586), human papillomavirus type 34 (NC_{—}001587), human papillomavirus type 4 (NC_{—}001457), human papillomavirus type 41 (NC_{—}001354), human papillomavirus type 48 (NC_{—}001690), human papillomavirus type 49 (NC_{—}001591), human papillomavirus type 5 (NC_{—}001531), human papillomavirus type 50 (NC_{—}001691), human papillomavirus type 53 (NC_{—}001593), human papillomavirus type 60 (NC_{—}001693), human papillomavirus type 63 (NC_{—}001458), human papillomavirus type 6b (NC_{—}001355), human papillomavirus type 7 (NC_{—}001595), human papillomavirus type 71 (NC_{—}002644), human papillomavirus type 9 (NC_{—}001596), human papillomavirus type 92 (NC_{—}004500), human papillomavirus type 96 (NC_{—}005134), human parainfluenza virus 1 (NC_{—}003461), human parainfluenza virus 2 (NC_{—}003443), human parainfluenza virus 3 (NC_{—}001796), human parechovirus (NC_{—}001897), human parvovirus 4 (NC_{—}007018), human parvovirus B19 (NC_{—}000883), human respiratory syncytial virus (NC_{—}001781), human rhinovirus A (NC_{—}001617), human rhinovirus B (NC_{—}001490), human spumaretrovirus (NC_{—}001795), human T-lymphotropic virus 1 (NC_{—}001436), human T-lymphotropic virus 2 (NC_{—}001488).

In certain examples, the target protein is produced from a nucleic acid sequence (e.g., genomic target nucleic acid sequence) from an oncogenic virus, such as Epstein-Barr Virus (EBV) or a Human Papilloma Virus (HPV, e.g., HPV16, HPV18). In other examples, the target protein produced from a nucleic acid sequence (e.g., genomic target nucleic acid sequence) is from a pathogenic virus, such as a Respiratory Syncytial Virus, a Hepatitis Virus (e.g., Hepatitis C Virus), a Coronavirus (e.g., SARS virus), an Adenovirus, a Polyomavirus, a Cytomegalovirus (CMV), or a Herpes Simplex Virus (HSV).

Additional specific examples of truncated proteins include, but are not limited to:

Parkinson Disease: The parkin gene may cause a form of autosomal recessive juvenile Parkinson disease due to a mutation in the parkin protein. This genetic mutation may be one of the most commonly known genetic causes of early-onset Parkinson disease. In a mouse model of Parkinson disease the parkin mutation results in a truncated parkin protein.

Crohn's Disease: The NOD2 gene (nucleotide-binding oligomerization domain 2) is one of the major susceptibility genes for Crohn's disease. The three main NOD2 mutations result in a truncated protein that predisposes affected individuals to Crohn's disease.

Breast Cancer: It is estimated that mutations in BRCA1 alone account for 50% of all familial, early-onset female breast cancers. Some of these mutations result in truncated BRCA1 protein. Truncation of the BRCA1 protein caused by inherited mutations in breast cancer-prone families is one possible mechanism of cancer induction that is currently under investigation.

Cardiac Sarcoidosis: Isolated cardiac sarcoidosis has been associated with the expression of a splice variant coding for a truncated BTNL2 protein.

Truncated forms of HER2/neu are constitutively expressed resulting in overexpression of trunctated HER2/neu. This overexpression is associated with several different forms of epithelial cancers of which breast cancer has been the most extensively studied.

Another member of the HER family is Epidermal Growth Factor Receptor (EGFR or HER1). Truncated forms of EGFR have been implicated in several types of epithelial cancers. Truncated EGFR has been closely linked with a type of brain tumor known is glioblastoma. Overexpression of Epidermal Growth Factor Receptor (EGFR, HER1) has been implicated in several types of epithelial cancers, including breast, colon, lung, and head and neck. The mechanism of EGFR overexpression and activation currently is being investigated. One mechanism of EGFR activation that has been associated with a high percentage of glioblastomas is truncated EGFR (EGFR Variant 3). This truncated form of EGFR does not require ligand binding for activation. Rather it is constitutively activated in the absence of any ligand binding such that cells expressing this aberrant form of EGFR are highly susceptible to malignant transformation. Truncated EGFR in tissues can be identified by immunohistochemistry according to the methods of this invention. A useful pair of antibodies for this purpose includes a first antibody (Pr1) 31G7, available from Invitrogen, that recognizes an epitope on the native EGFR protein and a second antibody (Pr2) E2451 from Spring Bioscience that recognizes an epitope on both the native and truncated EGFR protein. The 31G7 is a mouse monoclonal antibody and the E2451 is an epitope-specific rabbit polyclonal antibody. Truncated WT1 has been associated with various leukemias and truncated STAT has been linked to a specific leukemia known as acute myeloid leukemia.

Several other truncated oncoproteins have been identified, but the significance of these truncated forms is currently unknown. Some of the genes transcribing these oncoproteins are cph, fos, bax, bid, and ras.

C. Determining Epitopes that Distinguish Native and Truncated Proteins

Native proteins have at least one amino acid, more likely plural amino acids in a sequence, that are not present on the truncated protein, and which may provide epitopic portions that also are not present on the truncated protein. Certain disclosed embodiments of the present invention involve determining a first epitope (Ep1) found on the native protein and at a second epitope (Ep2) that is found on both the native protein and the truncated protein. A sample potentially comprising the native protein, the truncated protein, or both, is treated with at least two specific binding moieties, such as primary antibodies. A first primary antibody recognizes an epitope solely on the native protein. A second primary antibody recognizes an epitope on both the native protein and the truncated version of the native protein.

A collection of in vivo and in vitro methodologies are useful for epitope mapping, including binding assay, ELISPOT, HLA transgenic mice and prediction software. Epitope identification is exemplified herein by reference to HER2, a protein involved in regulating cell growth and differentiation. Over-expression of HER2 has been implicated as a contributing factor in certain types of cancers, including breast cancer. Some patients likely have a truncated form of HER2. New drugs have been developed that result in cancer regression. Patients with the truncated version of the HER2 protein may not benefit from treatment using these new drugs. Current methodologies do not distinguish between native and truncated forms of HER2.

HER2 includes an external domain having a first epitopic region (EP1). This first epitopic region is solely present on the native HER2 protein. HER2 also includes an internal domain having an epitopic portion that is present on both the native protein and truncated proteins.

A person of ordinary skill in the art will appreciate that HER2 simply exemplifies proteins that exist as both native protein and as a truncated form, or forms, thereof. Genetic deletions that result in truncated proteins can be identified by PCR or Southern blotting. These techniques do not always predict how much altered protein, if any, will be produced. Monoclonal antibodies (mAbs) can be used to identify target epitopes. For example, monoclonal antibodies can be mapped to particular specific exons. One technique involves generating random “libraries” of expressed protein fragments that are produced by cloning digestion fragments of a cDNA, such as may be produced by DNAseI, into an expression vector. The libraries are then used to locate epitopes recognized by monoclonal antibodies to fragments of amino acids within the protein fragment used to produce the antibodies. For example, a Duchenne patient with a frameshift deletion of exons 42 and 43 makes a truncated dystrophin encoded by exons 1-41. This can be detected by monoclonal antibodies up to and including those specific for exon 41 epitopes but not by monoclonal antibodies specific for exon 43 or later epitopes.

A general method utilizing immunohistochemistry to identify a first epitope (Ep1) and at least a second epitope (Ep2) on various targets is summarized below. A person of ordinary skill in the art will appreciate that other techniques also can be used to identify such epitopes, and that the scope of the present invention is not limited to this general method.

Tissue, such as cancer tissue, suspected of containing either native or truncated protein is fixed in 10% neutral buffered formalin. Following fixation the tissue is embedded into a paraffin block using standard histologic methods. Tissue slices of a suitable thickness, such as approximately 4 μm, are removed from the paraffin block using a microtope and fixed on a glass microscope slide. The microscope slide and attached tissue may be stored indefinitely until such time as testing commences.

At the time of testing, paraffin is removed from the tissue sample and the tissue is rehydrated using standard histologic methods. Antigen retrieval optionally may be performed. One antigen retrieval method comprises heating the tissue in an antigen retrieval solution for about 20 minutes at a temperature of about 95-121° C. This antigen retrieval step is required for certain epitopes that are masked by the fixation process. Not all epitopes are masked, and these epitopes do not require the antigen retrieval step. Tissues are then optionally treated with a reagent to inactivate endogenous enzymes.

Tissues are then incubated with a mixture of Pr1 and Pr2. Incubation occurs for a sufficient time to allow Pr1 and Pr2 to bind to Ep1 and Ep2, respectively, if present. Pr1 and Pr2 may be antibodies formed in different animal species such as rabbit and mouse respectively for Pr1 and Pr2. Unbound Pr1 and Pr2 are removed by rinsing the tissue in a buffer solution such as phosphate buffered saline.

The tissues are next incubated with a mixture of Se1 and Se2 for a sufficient time to allowing binding of Se1 and Se2 to Pr1 and Pr2, respectively, if present. Se1 is a secondary antibody that contains an enzyme En1. Se2 is a secondary antibody that contains a second enzyme En2. For example Se1 may be an anti-rabbit immunoglobulin conjugated to peroxidase, and Se2 may be an anti-mouse immunoglobulin conjugated to alkaline phosphatase. Unbound Se1 and Se2 are removed by rinsing the tissue in a buffer solution.

The tissue is reacted with the substrate of En1 for a sufficient time to form a colored reaction product on the tissue at the site where En1 is localized. For example, the substrate for peroxidase may be diaminobenzidine. Unreacted substrate is removed by rinsing the tissue with a buffer solution.

The tissue is reacted with a substrate of En2 for a sufficient time to form a second colored reaction product on the tissue at the site where En2 is localized. For example, the substrate for alkaline phosphatase may be Fast Red. Unreacted substrate is removed by rinsing the tissue with a buffer solution.

The tissue optionally may be stained with a counterstain, such as hematoxylin.

The tissue is covered with a coverslip in preparation for microscopic analysis. The tissue is analyzed, either manually by a trained microscopist, or automatically by a digital microscope coupled with computer aided image analysis.

### D. Distinguishing Different Epitopes

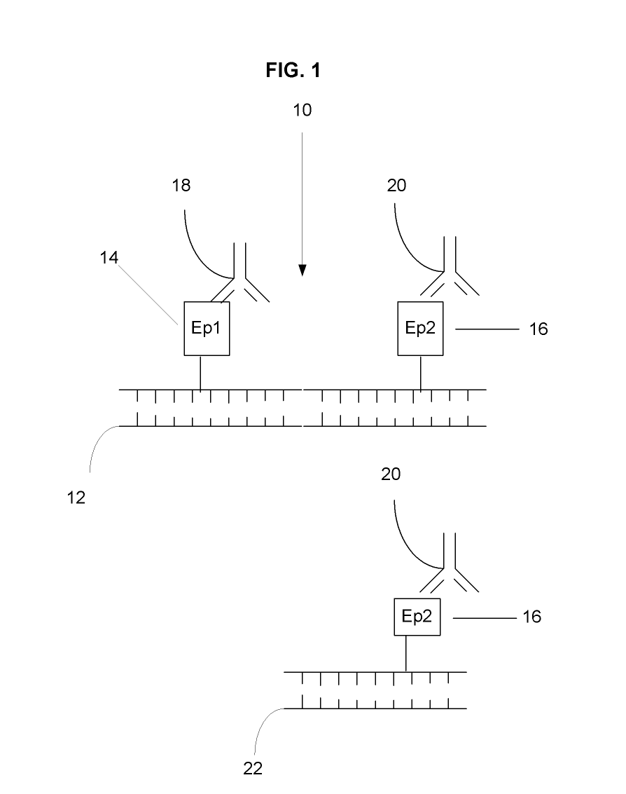

Once at least a first and second epitope have been identified that allow distinguishing between native and truncated forms of a protein, according to one disclosed embodiment a sample that is believed to include a truncated protein is treated with a first primary antibody that recognizes Ep1 and a second primary antibody that recognizes Ep2. This is illustrated schematically in FIG. 1. Sample 10 includes native protein 12 and truncated protein 22. Native protein 12 includes both first epitope 14 and second epitope 16. Truncated protein 22 includes only second epitope 16. Sample 10 is treated with a first antibody 18 that recognizes first epitope 14. Sample 10 also is treated with a second antibody 20 that recognizes second epitope 16. The first and second antibodies may be monoclonal antibodies. Sample 10 can be treated sequentially with antibody 18 and antibody 20. Alternatively, sample 10 can be treated with a mixture comprising antibody 18 and antibody 20. First epitope 14 and second epitope 16 are recognized by different primary antibodies 18 and 20, such that the primary antibodies are bound by the different epitopes.

The epitope/primary antibody binding pair must now be detected. A person of ordinary skill in the art will recognize that this can be accomplished a number of ways.

### E. Signal Generating Moiety Directly Conjugated to Primary Antibody

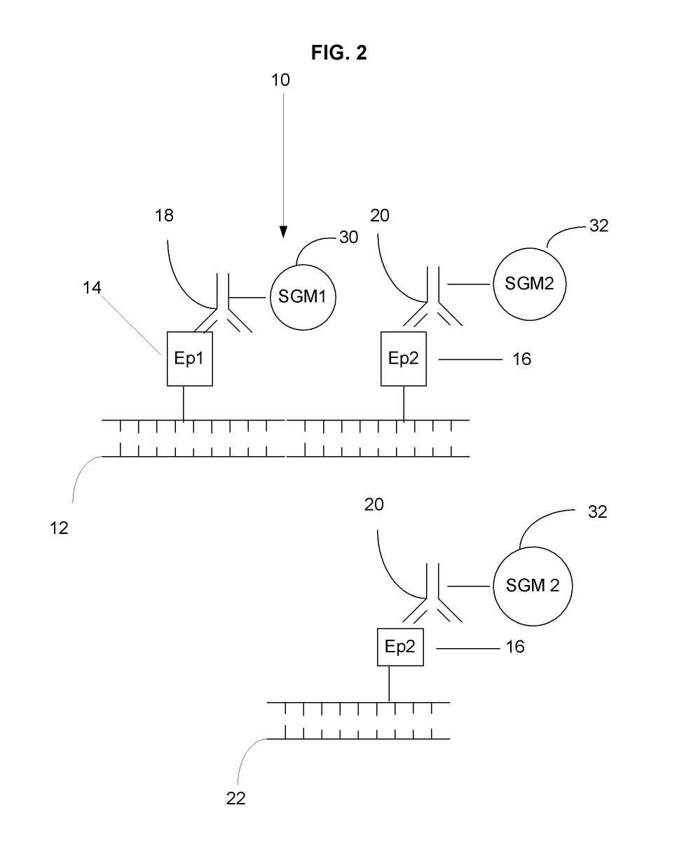

A first direct detection method is illustrated in FIG. 2. Sample 10 includes native protein 12 and truncated protein 22. Native protein 12 includes both first epitope 14 and second epitope 16. Truncated protein 22 includes only second epitope 16. Sample 10 is treated with a first antibody 18 that recognizes first epitope 14. Sample 10 also is treated with a second antibody 20 that recognizes second epitope 16. According to this first method, a first signal generating moiety 30 is conjugated to the first primary antibody 18. A second signal generating moiety 32 is conjugated to the second primary antibody 16. By using two different signal generating moieties, a binding pair comprising a first primary antibody 18 bound to first epitope 14 can be distinguished from a second binding pair comprising a second primary antibody 20 bound to second epitope 16.

Signal generating moieties are discussed herein in further detail, as well as certain prior patents and/or patent applications that are incorporated herein by reference. A person of ordinary skill in the art will appreciate that the signal generating moiety can be any of the variety of signal generating moieties disclosed herein or that otherwise would be known currently to a person of ordinary skill in the art, or here after developed, or combinations thereof. Examples of signal generating moieties include an enzyme, an organic chromophore, such as a flourphore, chromophoric nanoparticles, such as fluorescent quantum dots, etc. The signal generating moiety is used to visualize the complex.

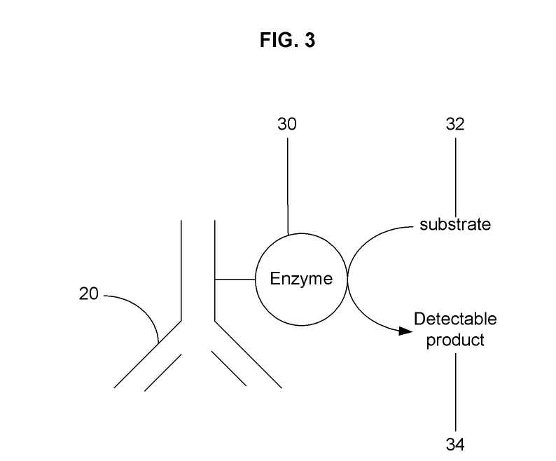

FIG. 3 illustrates one embodiment of a detection process using an enzymatic signal generating moiety. After formation of the binding pair, an enzyme substrate is provided. The enzyme-substrate reaction produces a detectable product 34. Thus, providing a substrate for the enzyme produces a uniquely identifiable reaction product, such as a colored precipitate.

### F. Secondary Anti-Antibodies

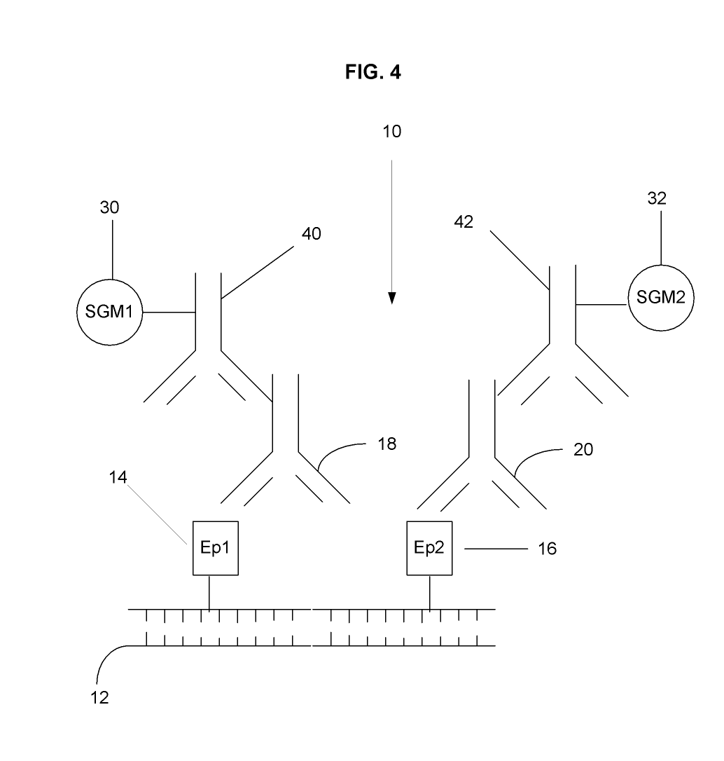

A signal amplification process also can be used. One embodiment of a signal amplification process is illustrated schematically in FIG. 4. This embodiment involves treating first and second primary antibodies that are bound to epitopes with first and second secondary antibodies that specifically bind to the first and second primary antibodies.

With reference to FIG. 4, sample 10 having a target protein 12 is selected. Native protein 12 is schematically illustrated in FIG. 4 as having a first epitope 14, Ep1, and a second epitope 16, Ep2. Sample 10 is treated with a first primary antibody 18 useful for detecting first epitope 14 on the target protein 12. Similarly, sample 10 can be treated with a second primary antibody 20 that specifically binds to a second epitope 16, Ep2. In one disclosed embodiment, a single sample is treated with both first antibody 18 and second antibody 20.

Sample 10 also is treated with a secondary antibody 40, which is an anti-antibody. For example, a primary antibody 18 might be from a first species, such as a mouse antibody. Secondary antibody can be from a second species, such as a rabbit or goat, and functions as an anti-mouse antibody. Secondary antibody 40 may be conjugated to a signal generating moiety 30, as discussed herein. Primary antibody 20 also can be specifically detected by second secondary antibody 42. Secondary antibody 42 also is conjugated directly to a signal generating moiety 32.

### G. Antibody-Hapten Conjugates

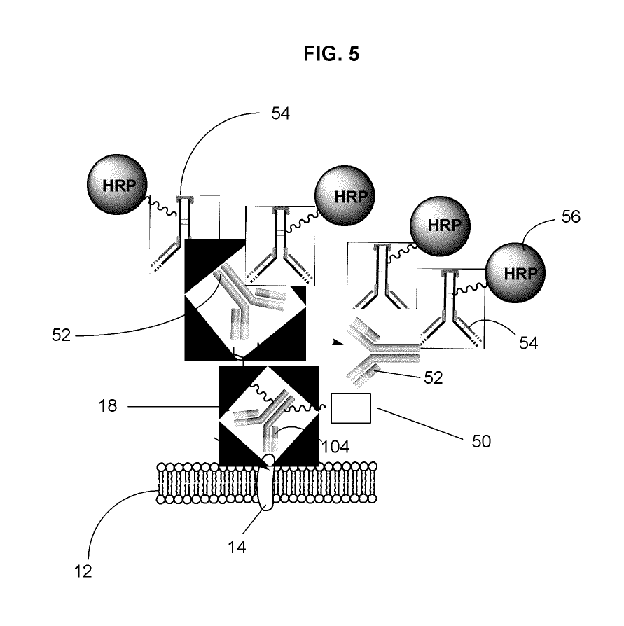

Certain embodiments of the present invention are facilitated by using anti-hapten monoclonal antibodies. FIG. 5 schematically represents one such embodiment. As with preceding examples, a sample 10 includes a particular target 14, such as a protein target, situated in a tissue 12. A primary antibody 18 directed to the target 14 is administered in a manner effective for the antibody to recognize the target. Primary antibody 18 has at least one hapten 50, and potentially plural haptens 50, conjugated thereto. A person of ordinary skill in the art will recognize first that the number of haptens conjugated to the antibody can vary. However, this number typically is from 1 to about 5 haptens, but more typically is 2 to 3. Furthermore, a person of ordinary skill in the art will appreciate that the haptens conjugated to the primary antibody can be the same or different.

Tissue sample 10 is treated with anti-hapten antibodies 52. For example, in the embodiment illustrated in FIG. 5, haptens 50, conjugated to the primary antibody 18, then effectively become coupled to an anti-hapten antibody 52. One disclosed embodiment now involves treating the tissue sample 10 with an antibody that recognizes the anti-hapten antibody. In the illustrated embodiment of FIG. 5, anti-antibodies 54 are conjugated to a signal generating moiety, such as an enzyme, including the illustrated horseradish peroxidase (HRP) enzymes 56. This complex is then incubated with an HRP substrate, as is known to persons of ordinary skill in the art, to form detectable, e.g. colored, precipitates.

To screen for anti-hapten monoclonal antibodies, a tissue sample, such as normal human tonsil tissue is obtained. The sample may be embedded in paraffin, and if so, the tissue sample is deparaffinized. Cell conditioning and antigen retrieval is then performed using, for example, VMSI CC1. A primary polyclonal antibody is conjugated to a hapten or haptens. Conjugation typically, but not necessarily, occurs at the Fc region of the antibody. Conjugating to the Fc region reduces the likelihood that the binding will affect the antibody specificity. A solution comprising an effective amount of the primary antibody is applied to the tissue for an effective period of time. Solely by way of example, the effective concentration may be about 10 μg/ml of the primary antibody, and the effective time period typically is about 60 minutes. The tissue sample is then washed. Thereafter, a potential anti-hapten antibody is applied to the tissue sample for an effective period of time, such as about 60 minutes. The antibody is then detected using any suitable means, such as, for example, VMSI Omni Map DAB stain.

### H. Exemplary Binding and Imaging Results

The preceding sections discussed general methods for specifically binding Ep1 and Ep2 in a sample, and then using signal generating moieties to uniquely identify a primary antibody-epitope conjugate. For a sample comprising a cell or tissue suspected of having a truncated protein that is reacted with the primary antibodies, the following results would obtain. First, if no protein is present then no primary antibodies would bind. If native protein is present then both the first (Pr1) and second (Pr2) primary antibodies would bind. If truncated protein is present then only the second primary antibody (Pr2) would bind.

The bound primary antibodies must then be visualized. For embodiments using secondary anti-antibodies that bind to the primary antibodies, the following results would obtain. First, if no target protein is present then no secondary antibodies would bind. If a native version of the target protein is present then a secondary antibody, conjugated to a first signal generating moiety, such as an enzyme, would bind to the first primary antibody Pr1 and as secondary antibody, conjugated to a second signal generating moiety, would bind to the second primary antibody Pr2. If truncated protein is present then only a secondary antibody conjugated to a second signal generating moiety would bind to second primary antibody Pr2.

The signal generating moieties are then used to visualize binding complexes. With reference to using enzymes as signal generating moieties, bound enzymes are visualized using substrates that react with the enzymes to produce specific colors as follows. If no protein is present then no substrates would react and no color would result. If truncated protein is present then the first signal generating moiety would produce a first result. For example, a first enzyme might be a peroxidase, and an appropriate substrate would be diaminobenzidine. The reaction of peroxidase and diaminobenzidine produces a chocolate color reaction. A second enzyme that might be commonly used for this purpose is alkaline phosphatase. A suitable substrate for alkaline phosphatase is Fast Red, which reacts with alkaline phosphatase to produce a red color at the site of the reaction. For these exemplary enzymes, if native protein is present then Fast Red would react with alkaline phosphatase and diaminobenzidine would react with peroxidase to produce a combination of red and chocolate that results in a brown color at the site of the reaction.

### III. HER2

The present invention is specifically exemplified with reference to HER2, a protein involved in regulating cell growth and differentiation. For HER2, a first useful primary antibody (Pr1) would be rabbit monoclonal antibody, clone SP3, to the HER2 external domain (Ep1), which is available from Spring Biosciences, Cat. No. M3034. A second useful primary antibody (Pr2) is mouse monoclonal antibody, clone SPM172, to the HER2 internal domain (Ep2), which also is available from Spring Biosciences, Cat. No. E4904. A cancer tissue suspected of having a truncated HER2 protein is treated with rabbit monoclonal antibody, clone SP3, to the HER2 external domain and mouse monoclonal antibody, clone SPM172, to the HER2 internal domain, either sequentially or simultaneously, in a manner effective to form antibody-epitope complexes. After incubation, excess unbound Pr1 and Pr2 antibodies are removed, such as by rinsing.

To determine if any primary antibodies bind to the target protein, secondary antibodies (Se1 and Se2) are applied to the sample, either as a mixture, or sequentially. A current embodiment contemplates using a mixture of two secondary antibodies. Certain secondary antibodies are provided as polymer conjugates. For example, a first polymer conjugate (Se1), available from BioCare Medical, Cat. No. RHRP520H, comprises a polymer backbone, anti-rabbit Ig secondary antibodies, and a peroxidase enzyme. A second polymer conjugate, also available from BioCare Medical, Cat. No. MALP521H, comprises a polymer backbone, anti-mouse Ig secondary antibodies, and alkaline phosphatase enzyme.

Excess unbound secondary antibodies are then rinsed off. If some of the secondary antibodies have bound to the primary antibodies, they remain bound to the sample. Also bound to the sample are then enzymes that are chemically attached to the secondary antibodies. The first secondary antibody (Se1) contains peroxidase (En1) and the second secondary antibody (Se2) contains alkaline phosphatase (En2).

The complexes must then be visualized. This can be achieved in this particular HER2 example using suitable enzyme substrates. For example, the sample can now be treated with diaminobenzidine (DAB; Spring Biosciences, Cat. No. DAB-015). If any peroxidase has bound, the DAB will produce a chocolate color. If no peroxidase has bound, the tissue will remain colorless. A suitable substrate for alkaline phosphatase is Fast Red, Spring Biosciences, Cat. No. LFR-015. After applying Fast Red, if any alkaline phosphatase is present the Fast Red produces a red color. If native HER2 is present, the Fast Red will be deposited on top of the previous DAB. The combination of these two colors results in an orange-brown color. If truncated HER2 is present, the Fast Red alone will be deposited on the tissue producing a red color. Stained slides may be viewed using a microscope to see which colors, and consequently which form of HER2, are present.

The exemplary HER2 embodiment uses secondary antibodies that react specifically with primary antibodies. Pr1 and Pr2 are primary antibodies that have been manufactured in different species of animals. For example Pr1 may be of rabbit origin. Rabbit primary antibodies are commonly used in research and diagnostic applications for detecting epitopes in tissues and cells. In this example Pr2 may be of mouse origin. Primary antibodies of mouse origin are also commonly used in research and diagnostic applications for detecting epitopes.

Secondary antibodies can be manufactured that bind specifically to mouse primary antibodies, and are known as anti-mouse immunoglobulins. Similarly, antibodies from other species can be used, such as antibodies that bind specifically to rabbit primary antibodies (anti-rabbit immunoglobulins).

The secondary antibodies can be further modified by directly or indirectly linking the secondary antibody with a signal generating moiety. In this exemplary embodiment, the signal generating moiety is an enzyme. Several different enzymes can be used but the most common ones for tissue staining include peroxidase and alkaline phosphatase. The method is facilitated by using two enzymes that generate two different color reactions.

### IV. Variations to Disclosed Embodiments

A person of ordinary skill in the art will appreciate that numerous modifications can be made to the exemplary disclosed embodiment that are within the scope of the present invention. For example, the primary antibodies could be: (1) made in different species other than mouse or rabbit, including goat; (2) chemically modified to be recognized by alternative secondary antibodies; (3) directly conjugated with a directable label, such as an enzyme; and/or (4) directly conjugated to different color fluorochromes.

With reference to the secondary antibodies, the disclosed embodiments could be: (1) made to react against different species other than mouse or rabbit; (2) chemically modified by linking multiple secondary antibodies to a single polymer backbone; (3) directly or indirectly conjugated to enzymes, including enzymes other than peroxidase alkaline phosphatase; and/or (4) conjugated to different colored signal generating moieties other than enzymes, such as fluorochromes.

With reference to enzyme substrates, such substrates could be: (1) different from Dab and Fast Red, particularly if different enzymes are used as signal generating moieties; and (2) eliminated completely if other signal generating moieties, such as fluorochromes, are used.

With reference to exemplary disclosed method steps, disclosed embodiments also could be modified in various ways and still be within the scope of the disclosed invention. For example, primary antibodies could be directly conjugated to a signal generating moiety, such as an enzyme, thereby eliminating the need to form a complex with the primary antibody with a binding pair that itself is conjugated to a signal generating moiety. And the primary antibody could be directly conjugated to a signal generating moiety other than an enzyme, such as a fluorochrome, thereby eliminating subsequent steps other than visualization. Also, the secondary antibody could be directly conjugated to a signal generating moiety, such as a fluorochrome, instead of an enzyme. Although these modifications are within the scope of the present invention, a loss of sensitivity may result. It currently is believed that a three-step immunohistochemistry labeling protocol provides the best sensitivity.

Antigen retrieval also can be used with disclosed embodiments. Antigen retrieval involves pre-treating tissue samples so that the tissues are more reactive with primary antibodies. Antigen retrieval is discussed in, for example, Ventana Medical System, Inc.'s U.S. Pat. No. 7,067,325, which is incorporated herein by reference.

Endogenous enzymes that could potentially interfere with the analysis also can be blocked. This can be accomplished, for example, by pre-treating samples to reduce or substantially eliminate endogenous enzymes, such as peroxidase or alkaline phosphatase.

Counterstaining is a method of post-treating the tissues after they have already been stained, such that their structures can be more readily visualized under a microscope. For example, a counterstain is optionally used prior to coverslipping to render the immunohistochemical stain more distinct. Counterstains differ in color from a primary stain. Numerous counterstains are well known, such as hematoxylin, eosin, methyl green, methylene blue, Geimsa, Alcian blue, and Nuclear Fast Red.

Certain aspects, or all, of the disclosed embodiments can be automated, and facilitated by computer analysis and/or image analysis system. In some applications it may be necessary to measure precise color ratios. For example, if both truncated protein and native protein are present together on a sample, the ratio of these proteins could be determined from the amount of color that represents each form. Certain disclosed embodiments involve acquiring digital images. This can be done by coupling a digital camera to a microscope. Digital images obtained of stained samples are analyzed using image analysis software. Color can be measured in several different ways. For example, color can be measured as red, blue, and green values; hue, saturation, and intensity values; by measuring a specific wavelength or range of wavelengths using a spectral imaging camera; and any and all combinations of such techniques.

One disclosed embodiment involves using brightfield imaging with chromogenic dyes. White light in the visible spectrum is transmitted through the dye. The dye absorbs light of certain wavelengths and transmits other wavelengths. This changes the light from white to colored depending on the specific wavelengths of light transmitted. The dye also may be a fluorogenic dye visualized using a fluorescence microscope.

### V. Signal Generating Moieties

Conjugates comprising signal generating moieties, such as conjugates of specific-binding moieties and signal-generating moieties, can be used in assays for detecting specific target molecules in biological samples. The signal-generating portion is utilized to provide a detectable signal that indicates the presence/and or location of the target. Examples of signal-generating moieties include, by way of example and without limitation: enzymes, such as horseradish peroxidase, alkaline phosphatase, acid phosphatase, glucose oxidase, β-galactosidase, β-glucuronidase or β-lactamase. Horseradish peroxidase is widely used as a label for immunoglobulins in many different immunochemistry applications including ELISA, immunoblotting and immunohistochemistry. In addition to other possible disclosed embodiments, HRP can be conjugated to antibodies by several different methods including glutaraldehyde, periodate oxidation, through disulfide bonds, and also via amino and thiol directed cross-linkers. HRP is the smallest and most stable of the three most popular enzyme labels (HRP, alkaline phosphatase, and B-galactosidase) and its glycosylation leads to lower non-specific binding; fluorescent molecules (such as fluoresceins, coumarins, BODIPY dyes, resorufins, rhodamines; additional examples can be found in The Handbook—A Guide to Fluorescent Probes and Labeling Technologies, Invitrogen Corporation, Eugene, Oreg.), detectable constructs (such as fluorescent constructs like quantum dots, which can be obtained, for example, from Invitrogen Corporation, Eugene, Oreg.; see, for example, U.S. Pat. Nos. 6,815,064, 6,682596 and 6,649,138, each of which patents is incorporated by reference herein), metal chelates (such as DOTA and DPTA chelates of radioactive or paramagnetic metal ions like Gd³⁺) and liposomes (such as liposomes sequestering fluorescent molecules.

When the signal-generating moiety includes an enzyme, a chromagenic compound, fluorogenic compound, or luminogenic compound can be used to generate a detectable signal (a wide variety of such compounds are available, for example, from Invitrogen, Eugene Oreg.). Particular examples of chromogenic compounds include di-aminobenzidine (DAB), 4-nitrophenylphospate (pNPP), fast red, bromochloroindolyl phosphate (BCIP), nitro blue tetrazolium (NBT), BCIP/NBT, fast red, AP Orange, AP blue, tetramethylbenzidine (TMB), 2,2′-azino-di-[3-ethylbenzothiazoline sulphonate] (ABTS), o-dianisidine, 4-chloronaphthol (4-CN), nitrophenyl-β-D-galactopyranoside (ONPG), o-phenylenediamine (OPD), 5-bromo-4-chloro-3-indolyl-β-galactopyranoside (X-Gal), methylumbelliferyl-β-D-galactopyranoside (MU-Gal), p-nitorphenyl-α-D-galactopyranoside (PNP), 5-bromo-4-chloro-3-indolyl-β-D-glucuronide (X-Gluc), 3-amino-9-ethyl carbazol (AEC), fuchsin, iodonitrotetrazolium (INT), tetrazolium blue and tetrazolium violet.

Labeled secondary antibodies can be purchased from a number of sources, such as, but not limited to, Pierce Co. Amersham and Evident Technologies provide a broad range of conjugated antibody possibilities. CyDye, EviTag Quantum Dot, fluorescein (FITC), and rhodamine can be utilized. These conjugates span a variety of applications, colors, and emission ranges. The EviTag Quantum Dots from Evident Technologies offer photo-stability and multicolor fluorescence in a variety of wavelengths, with the advantage over organic fluorophores of improved photostability, color multiplexing, and single source excitation. Each Evitag generates a sharp emission wavelength making them ideal for multiplexing in intact cell environments.

The Amersham CyDyes offer superior photostability over a broad range of pH values. For a tutorial on fluorescent markers, with the chemical structures of the labels, see: http://www.hmds.org.uk/fluorochrome.html. See the following link on how to label with haptens: http://probes.invitrogen.com/handbook/boxes/2020.html

One type of detectable conjugate is a covalent conjugate of an antibody and a fluorophore. Directing photons toward the conjugate that are of a wavelength absorbed by the fluorophore stimulates fluorescence that can be detected and used to qualitate, quantitate and/or locate the antibody. A majority of the fluorescent moieties used as fluorophores are organic molecules having conjugated pi-electron systems. While such organic fluorophores can provide intense fluorescence signals, they exhibit a number of properties that limit their effectiveness, especially in multiplex assays and when archival test results are needed.

Organic fluorophores can be photo-bleached by prolonged illumination with an excitation source, which limits the time period during which maximal and/or detectable signals can be retrieved from a sample. Prolonged illumination and/or prolonged exposure to oxygen can permanently convert organic fluorophores into non-fluorescent molecules. Thus, fluorescence detection has not been routinely used when an archival sample is needed.

Chromophoric and/or fluorescent semiconductor nanocrystals, also often referred to as quantum dots, can be used for identifying complexes. Nanocrystalline quantum dots are semiconductor nanocrystalline particles, and without limiting the present invention to use with particle light emitters of a particular size, typically measure 2-10 nm in size (roughly the size of typical proteins). Quantum dots typically are stable fluorophores, often are resistant to photo bleaching, and have a wide range of excitation, wave-length and narrow emission spectrum. Quantum dots having particular emission characteristics, such as emissions at particular wave-lengths, can be selected such that plural different quantum dots having plural different emission characteristics can be used to identify plural different targets. Quantum dot bioconjugates are characterized by quantum yields comparable to the brightest traditional dyes available. Additionally, these quantum dot-based fluorophores absorb 10-1000 times more light than traditional dyes. Emission from the quantum dots is narrow and symmetric, which means overlap with other colors is minimized, resulting in minimal bleed through into adjacent detection channels and attenuated crosstalk, in spite of the fact that many more colors can be used simultaneously. Symmetrical and tuneable emission spectra can be varied according to the size and material composition of the particles, which allows flexible and close spacing of different quantum dots without substantial spectral overlap. In addition, their absorption spectra are broad, which makes it possible to excite all quantum dot color variants simultaneously using a single excitation wavelength, thereby minimizing sample autofluorescence.

Furthermore, it has been found that pegylation, the introduction of polyethylene glycol groups onto the quantum dot, can substantially decrease non-specific protein:quantum dot interaction. Certain quantum dots are commercially available, such as from Quantum Dot Corp., of Hayward, Calif., and Invitrogen.

Standard fluorescence microscopes are an inexpensive tool for the detection of quantum dot bioconjugates. Since quantum dot conjugates are virtually photo-stable, time can be taken with the microscope to find regions of interest and adequately focus on the samples. Quantum dot conjugates are useful any time bright photo-stable emission is required and are particularly useful in multicolor applications where only one excitation source/filter is available and minimal crosstalk among the colors is required. For example, quantum dots have been used to form conjugates of Streptavidin and IgG to label cell surface markers and nuclear antigens and to stain microtubules and actin (Wu, X. et al. (2003). Nature Biotech. 21, 41-46).

As an example, fluorescence can be measured with the multispectral imaging system Nuanc™ (Cambridge Research & Instrumentation, Woburn, Mass.). As another example, fluorescence can be measured with the spectral imaging system SpectrView™ (Applied Spectral Imaging, Vista, Calif.). Multispectral imaging is a technique in which spectroscopic information at each pixel of an image is gathered and the resulting data analyzed with spectral image-processing software. For example, the Nuance system can take a series of images at different wavelengths that are electronically and continuously selectable and then utilized with an analysis program designed for handling such data. The Nuance system is able to obtain quantitative information from multiple dyes simultaneously, even when the spectra of the dyes are highly overlapping or when they are co-localized, or occurring at the same point in the sample, provided that the spectral curves are different. Many biological materials autofluoresce, or emit lower-energy light when excited by higher-energy light. This signal can result in lower contrast images and data. High-sensitivity cameras without multispectral imaging capability only increase the autofluorescence signal along with the fluorescence signal. Multispectral imaging can unmix, or separate out, autofluorescence from tissue and, thereby, increase the achievable signal-to-noise ratio.

Haptens can be conjugated to quantum dots, and quantum dot fluorescence can be stimulated, such as by using fluorescence resonance energy transfer (FRET) whereby low-wavelength light stimulates quantum dot fluorescence. Invitrogen has determined that biotin-conjugated quantum dots had a 100-fold lower limit of detection for the biotin derivative biocytin than anti-biotin Alexa Fluor. Fully biotinylated quantum dots were 10-fold less sensitive than quantum dots with 25 percent biotin coverage.

Quantum dot use has so far been limited by their lack of biocompatibility. New advances in surface coating chemistry, however, have helped to overcome these problems. See, for example, Wu, X. et al Immunofluorescent labeling of cancer marker HER2 and other cellular targets with semiconductor quantum dots, Nature Biotechnol. 21, 41-46 (2003); Jaiswal, J. K., Mattoussi, H., Mauro, J. M. & Simon, S. M. Long-term multiple color imaging of live cells using quantum dot bioconjugates, Nature Biotechnol. 21, 47-51 (2003); and Dubertret, B. et al. In vivo imaging of quantum dots encapsulated in phospholipid micelles. Science 298, 1759-1762 (2002).

Quantum dots also have been conjugated to biorecognition molecules, Id., such as streptavidin. These conjugates have been used on both fixed cells and tissue sections. In addition, cell-surface proteins and the endocytic compartments of live cells have been labeled with quantum dot bioconjugates.

Fluorescent proteins also can be used as a carrier, or can be coupled to a carrier, to facilitate visualization. For example, green fluorescent protein (GFP) was originally isolated from the light-emitting organ of the jellyfish Aequorea victoria. Chimeric GFP fusions can be expressed in situ by gene transfer into cells, and can be localized to particular sites within the cell by appropriate targeting signals. Spectral variants with blue, cyan and yellowish-green emissions have been successfully generated from the Aequorea GFP, but none exhibit emission maxima longer than 529 nm. GFP-like proteins have been isolated from Anthozoa (coral animals) that significantly expanded the range of colors available for biological applications. The family of ‘GFP-like proteins’ deposited in sequence databases now includes approximately 30 significantly different members. Fluorescent proteins refers to proteins that can become spontaneously fluorescent through the autocatalytic synthesis of a chromophore.

Proteins that fluoresce at red or far-red wavelengths (red fluorescent proteins or RFPs) are known. RFPs can be used in combination with other fluorescent proteins that fluoresce at shorter wavelengths for both multicolor labeling and fluorescence resonance energy transfer (FRET) experiments. Commercially available RFPs are derived from two wild-type GFP-like proteins. DsRed (drFP583) has excitation and emission maxima at 558 nm and 583 nm, respectively. A far-red fluorescent protein was generated by mutagenesis of a chromoprotein that absorbs at 571 nm. HcRed1 (Clontech) has excitation and emission maxima at 588 nm and 618 nm, respectively. The fluorescent protein that emits fluorescence at the longest wavelength (without any mutations being introduced) is eqFP611, cloned from the sea anemone Entacmaea quadricolor. This protein absorbs at 559 nm and emits at 611 nm. As many spectral variants have emerged, more investigators are becoming interested in the simultaneous imaging of multiple fluorophores and/or FRET signals.

Fusion proteins also can be used to form hapten conjugates of the present invention. There are at least three points to consider when creating a functional fluorescent protein: the fluorescent protein must fold correctly to fluoresce; the host protein must fold correctly to be functional; and the integrity of the chimeric protein must be maintained.

The length and sequence of any linker between the fluorescent protein and host protein should be optimized for each specific application. The most widely used linker designs have sequences that primarily consist of glycine (Gly) and serine (Ser) stretches, Ser residues being interspersed to improve the solubility of a poly-Gly stretch.

The decision of whether to fuse a fluorescent protein to the amino or carboxyl terminus of a protein depends on the properties of the protein. For example, a particular terminus might need to be preserved to retain proper protein function or to ensure correct localization. This decision might also be made on the basis of structural aspects of the particular fluorescent protein. For example, Aequorea GFP has a floppy carboxyl terminal tail of approximately ten amino acids, which makes its fusion to the amino terminus of other proteins possible without the addition of a linker. By contrast, DsRed is more successfully fused to the carboxyl terminus of proteins of interest, because the amino termini project fully from a tetrameric complex of DsRed. If neither end of a host protein can be modified, it is possible to insert the fluorescent protein into the middle of the protein.

Citrine and Venus, two bright versions of a yellow-emitting mutant of GFP (YFP) that mature efficiently, have recently been developed.

Two recently developed varieties of DsRed, known as T1 and E57, display improved maturation, making them preferable for use in dual-color experiments.

Fluorescence of some GFP variants can be ‘photoactivated’ by specific illumination, which provides the advantage that fluorescence can be turned on at a chosen time point. Three fluorescent proteins that undergo photochemical modification in or near the chromophore have been developed, PA-GFP, Kaede and KFP1, that enable selective activation of fluorescence signals after specific illumination, and can be used to fluorescently mark individual cells, organelles or proteins.

Table 1 provides additional examples of signal generating moieties and conjugates comprising such moieties.

TABLE 1
Exemplary Antibody-Signal generating moiety Conjugates

| | | | Label | Label |
|---|---|---|---|---|
| Antibody Conjugate | | Emitted | Excitatio | Emissio |
| Label | Recommended for . . . | Color | n (nm) | n (nm) |
| Lake Placid Blue | Flow cytometry, immunoblots, | Blue | <450 | 490 |
| (EviTag ™ Quantum Dot) | and fluorescent microscopy | | | |
| Fluorescein | Flow cytometry, incl. BD FACS | Dark | 494 | 518 |
| (i.e. FITC) | systems and Guava System, and | Green | | |
| | fluorescent microscopy | | | |
| Adirondack Green | Flow cytometry, immunoblots, | Dark | <450 | 520 |
| (EviTag ™ Quantum Dot) | and fluorescent microscopy | Green | | |
| Rhodamine Green | Fluorescent microscopy | Dark | 502 | 527 |
| | | Green | | |
| Catskill Green | Fluorescent microscopy | Dark | <450 | 540 |
| (EviTag ™ Quantum Dot) | | Green | | |
| Rhodamine 6G | Flow cytometry, immunoblots, | Light | 525 | 555 |
| | and fluorescent microscopy | Green | | |
| Hops Yellow | Flow cytometry, immunoblots, | Light | <450 | 560 |
| (EviTag ™ Quantum Dot) | and fluorescent microscopy | Green | | |
| Amersham Cy3 | Fluorescent microscopy | Light | 550 | 565 |
| | | Green | | |
| R-Phycoerythrin (PE) | Flow cytometry, Luminex ® and | Yellow | (495)56 | 575 |
| | Guava systems, FRET assays, | | 5 | |
| | and capillary electrophoresis; use | | | |
| | with FITC for double labeling | | | |
| Rhodamine Red | Flow cytometry, fluorescent | Light | 560 | 580 |
| | microscopy | Orange | | |
| Birch Yellow | Fluorescent microscopy | Light | <450 | 580 |
| (EviTag ™ Quantum Dot) | | Orange | | |
| Amersham Cy3.5 | Fluorescent microscopy | Dark | 581 | 596 |
| | | Orange | | |
| Fort Orange | Flow cytometry, immunoblots, | Dark | <450 | 600 |
| (EviTag ™ Quantum Dot) | and fluorescent microscopy | Orange | | |
| SulfoRhodamine | Flow cytometry and fluorescent | Light | 596 | 615 |
| (Alias Texas Red ®) | microscopy | Red | | |
| Amersham Cy5 | Immunoblot, incl.. Amersham | Medium | 650 | 670 |
| | Typhoon System, and | Red | | |
| | immunofluorescent applications | | | |
| Allophycocyanin (APC) | FRET assays and HTRF assays | Medium | 652 | 670 |
| | | Red | | |
| Amersham Cy5.5 | Immunoblot, especially LI-COR | Dark | 675 | 694 |
| | Odyssey systems | Red | | |
| Biotin | Flow cytometry and other | | — | |
| | fluorescent applications | | | |

Many of these labels can be used with multiple antibodies that do not cross-react to create custom
multiplexed assays.

### VI. Test Kits

Disclosed embodiments of the present invention provide, in part, kits for carrying out various embodiments of the method of the invention. Certain disclosed test kit embodiments comprise a first specific binding moiety for detecting a first specific binding pair on a native form of a protein, and a second specific binding moiety for detecting a second binding pair on both the native form of the protein and on a truncated form of the protein. For example, kits within the scope of the present invention can include first and second primary antibodies for recognizing first and second epitopes on a native form of the protein and only one of the first and second epitopes on the truncated form of the protein. The kit also may comprise first and second secondary anti-primary antibodies. Further, the first and second secondary anti-primary antibodies may be coupled to signal generating moieties. If the signal generating moieties are enzymes, then the kit may further comprise substrates for reaction with the enzymes. Disclosed kit embodiments can include additional components, including but not limited to plural additional antibodies. Such kits may be used, for example, by a clinician or physician.

### VII. Prior Patents and Applications Incorporated by Reference

The following applications and patents are commonly owned, or shall be commonly owned, with the present application, and are incorporated herein by reference: U.S. Pat. No. 5,414,138; U.S. patent application Ser. No. 11/982,627, filed Nov. 1, 2007, entitled Haptens, Hapten Conjugates, Compositions Thereof and Method for Their Preparation and Use; U.S. provisional application No. 60/931,546, filed on May 23, 2007, entitled Polymeric Hapten Carriers as Signal Amplifiers for Immunohistochemistry and In Situ Hybridization; Antigen Retrieval Methods for Immunohistochemistry, filed as a provisional application on Dec. 28, 2007; and Microwave Antigen Retrieval in Non-Aqueous Solvents, filed as a provisional application on Dec. 28, 2007.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. I therefore claim as my invention all that comes within the scope and spirit of the following claims.

1. A method for detecting a truncated molecule, comprising:
providing a sample potentially comprising a native molecule and/or a truncated molecule, the native molecule comprising at least first and second regions recognized by first and second specific binding pairs, the truncated molecule including only one of the first and second regions;
applying a composition to the sample comprising the first and second binding pairs; and
detecting bound first and second binding pairs.

2. (canceled)

3. The method according to claim 1 where the molecule is a protein.

4. The method according to claim 1 where the molecule is a protein having a first epitope and a second epitope, and where the method further comprises treating the sample with at least two primary antibodies in a manner effective to form epitope-antibody complexes, a first primary antibody recognizing a first epitope on a truncated molecule and a second primary antibody recognizing a second epitope on the native molecule and on the truncated molecule.

5. The method according to claim 4, further comprising treating the sample with first and second secondary antibodies that recognize the first and second primary antibodies.

6. The method according to claim 5 where the first and second secondary antibodies are effectively coupled to signal generating moieties.

7. The method according to claim 6 where the signal generating moieties are first and second enzymes.

8. The method according to claim 7 further comprising treating the sample with a first substrate for the first enzyme and a second substrate for the second enzyme, wherein reactions between enzyme and substrate produces detectable reactions.

9. (canceled)

10. The method according to claim where detecting comprises using signal generating moieties selected from enzymes, chromophores, quantum dots, or combinations thereof.

11. The method according to claim where the signal generating moiety is an enzyme selected from peroxidase, alkaline phosphatase, acid phosphatase, glucose oxidase, β-galactosidase, β-glucuronidase or β-lactamase.

12-13. (canceled)

14. The method according to claim 5 where the antibodies are monoclonal antibodies from different species.

15. (canceled)

16. The method according to claim 1 where the sample includes a biological target molecule that expresses at least two epitopes that can be specifically bound by primary antibodies, a first epitope being present on a truncated molecule form, and the first and a second epitope being present on a native form of the molecule.

17. (canceled)

18. The method according to claim 3 where the protein is EGFR, HER1, HER2, HER2/neu), parkin protein, truncated proteins produced from NOD2 mutations, truncated proteins produced from BRCA1 mutations, and truncated BTNL2 protein

19. The method according to claim 18 where HER2 includes an external domain having a first epitopic region solely present on a native HER2 protein, and an internal domain having an epitopic portion that is present on both the native protein and a truncated HER2 protein.

20. (canceled)

21. The method according to claim 19 comprising treating the sample with first and second primary antibodies from a first species, and where the method further comprises treating the first and second primary antibodies that are bound to epitopes with first and second secondary anti-antibodies that specifically bind to the first and second primary antibodies.

22. (canceled)

23. The method according to claim 21 where the first and second secondary anti-antibodies are coupled to signal generating moieties.

24. The method according to claim 21 where the first and second primary antibodies have at least one hapten conjugated thereto, and the method comprises treating the sample with anti-hapten antibodies.

25-28. (canceled)

29. The method according to claim 19 where if native HER2 protein is present then both first and second primary antibodies bind, and if truncated HER2 protein is present then only the second primary antibody binds.

30-32. (canceled)

33. The method according to claim 1 further comprising performing antigen retrieval to facilitate reaction with primary antibodies.

34. The method according to claim 1 further comprising blocking or substantially eliminating endogenous enzyme or enzymes that potentially interfere with the analysis.

35. (canceled)

36. The method according to claim 1 further comprising counterstaining with hematoxylin, eosin, methyl green, methylene blue, Geimsa, Alcian blue, and Nuclear Fast Red.

37-38. (canceled)

39. A method for detecting a truncated protein molecule, comprising:
providing a sample potentially comprising a native protein and/or a truncated protein, the native protein comprising at least first and second epitopes recognized by first and second antibodies, the truncated molecule including only one of the first and second epitopes;
optionally pretreating the sample to facilitate reaction with primary antibodies;
applying a composition comprising the first and second antibodies to the sample;
detecting bound first and second primary antibodies; and
optionally counterstaining the sample.

40. (canceled)

41. The method according to claim 39 where the first and second primary antibodies are coupled to first and second signal generating moieties selected from enzymes, chromophores, quantum dots, or combinations thereof.

42. The method according to claim 39, further comprising treating the sample with first and second secondary antibodies that recognize the first and second primary antibodies.

43. The method according to claim 42 where the first and second secondary antibodies are effectively coupled to signal generating moieties.

44. The method according to claim 43 where the signal generating moieties are first and second enzymes, and the method further comprises treating the sample with a first substrate for the first enzyme and a second substrate for the second enzyme, wherein reactions between enzyme and substrate produces detectable reactions.

45. The method according to claim 44 where the enzymes are peroxidase, alkaline phosphatase, acid phosphatase, glucose oxidase, β-galactosidase, β-glucuronidase or β-lactamase.

46. (canceled)

47. The method according to claim 42 where the antibodies are monoclonal antibodies from different species.

48. (canceled)

49. The method according to claim 42 comprising treating the sample with first and second primary antibodies from a first species, and where the method further comprises treating the first and second primary antibodies that are bound to epitopes with first and second secondary anti-antibodies that specifically bind to the first and second primary antibodies.

50. The method according to claim 39 where the first and second primary antibodies have at least one hapten conjugated thereto, and the method further comprises treating the sample with anti-hapten antibodies.

51. The method according to claim 50 where the anti-hapten antibodies are coupled to signal generating moieties.

52-53. (canceled)

54. A method for detecting a truncated HER2 protein, comprising:
providing a tissue sample potentially including a native HER2 protein, a truncated HER2 protein, or both;
incubating the tissue sample with at least two primary antibodies, a first antibody recognizing a first epitope on a native HER2 protein and a second antibody recognizing a second epitope on both the native HER2 protein and the truncated form of the HER2 protein;
treating the sample with first and second secondary antibodies that specifically bind to the first and second primary antibodies, the first and second secondary antibodies being effectively coupled to enzymes useful as signal generating moieties;
applying suitable enzyme substrates; and
detecting color changes to determine if native HER2 is present, truncated HER2 is present, or both.

55. The method according to claim 54 where the enzymes are peroxidase and alkaline phosphatase, and the substrates are diaminobenzidine as a substrate for peroxidase, which produces a chocolate color reaction, and Fast Red as a substrate for alkaline phosphatase, which produces a red color.

56. The method according to claim 54 where the first primary antibody is rabbit monoclonal antibody, clone SP3, to the HER2 external domain, and the second primary antibody is mouse monoclonal antibody, clone SPM172, to the HER2 internal domain.

57. The method according to claim 56 where secondary antibodies are provided as polymer conjugates.

58. The method according to claim 57 where a first polymer conjugate comprises a polymer backbone, anti-rabbit Ig secondary antibodies, and peroxidase, and a second polymer conjugate comprises a polymer backbone, anti-mouse Ig secondary antibodies, and alkaline phosphatase.

59. A test kit for detecting truncated proteins, comprising at least two specific binding moieties, a first specific binding moiety for detecting a first specific binding pair on a native form of a protein, and a second specific binding moiety for detecting a second binding pair on both the native form of the protein and on a truncated form of the protein.

60-67. (canceled)
